(19) United States
(12) Patent Application Publication
(10) Pub. No.: US 2024/0066203 A1
(43) Pub. Date: Feb. 29, 2024

(54) EAR CLEANING DEVICE

(71) Applicant: SHENZHEN INTELLIGENT DOCTOR TECHNOLOGY CO., LTD, SHENZHEN (CN)

(72) Inventor: QINGQUAN LIU, SHENZHEN (CN)

(21) Appl. No.: 18/055,424

(22) Filed: Nov. 15, 2022

(30) Foreign Application Priority Data
Aug. 26, 2022 (CN) 202222267785.1
Aug. 31, 2022 (CN) 202222328025.7

(51) Int. Cl. A61M 3/02

(52) U.S. Cl. CPC A61M 3/0202; A61M 3/0258; A61M 3/0279

(57) ABSTRACT

An ear cleaning device relating to a technology of medical auxiliary equipment is provided, the ear cleaning device includes a bottle body, a spray assembly, and a temperature display assembly arranged on the bottle body. The temperature display assembly includes a temperature sensor and a display screen electrically connected with the temperature sensor, The temperature sensor is arranged in the bottle body and is configured to detect temperature of liquid in the bottle body, and the display screen is configured to display the temperature. The spray assembly is connected to a nozzle and a straw, and the straw extends into the bottle body. The ear cleaning device displays the temperature of the current liquid in real time, which facilitates the control of the temperature of the liquid during a washing process.

### CROSS REFERENCE TO RELATED APPLICATION

This patent application claims the benefit and priority of Chinese Patent Application No. 202222267785.1, entitled “manual ear cleaning device” and filed on Aug. 26, 2022, and Chinese Patent Application No. 202222328025.7, entitled “electric ear cleaning device” and filed on Aug. 31, 2022, the disclosures of which are incorporated by reference herein in their entirety as part of the present disclosure.

### TECHNICAL FIELD

The present disclosure relates to a technical field of medical auxiliary equipment, and in particular to an ear cleaning device.

### BACKGROUND

External auditory canal is a curved tube from an external auditory door to an eardrum, a total length of which is about 2.1 to 2.5 cm. Secretion produced by glands in cartilage skin of the external auditory canal is earwax, and human earwax may affect hearing, when using an unclean ear pick to remove, diseases of the external auditory canal may be caused, main manifestations of which include different kinds of inflammation, and clinical manifestations include ear itching, ear discomfort, ear congestion, ear swelling, and/or watery discharge. Hearing may be seriously affected when the inflammation is severe or the secretion blocks the auditory canal.

Currently, an ear cleaning device is usually used for cleaning the external auditory canal, and when cleaning ears, warm saline, hydrogen peroxide, or sodium bicarbonate may be chosen for cleaning according to personal situation, since the auditory canal is narrow and small and the eardrum is sensitive, when treating or cleaning the auditory canal, if the temperature of the liquid is too high or too low, patients may easily feel discomfort.

### SUMMARY

The present disclosure provides an ear cleaning device, which may display temperature of current liquid in real time and is convenient for the control of the temperature of the liquid during a cleaning process.

Embodiments of the present disclosure may be achieved as following.

The present disclosure provides an ear cleaning device including a bottle body, a spray assembly, and a temperature display assembly. The spray assembly and the temperature display assembly are arranged on the bottle body. The temperature display assembly includes a temperature sensor and a display screen, the display screen is electrically connected with the temperature sensor. The temperature sensor is arranged in the bottle body and is configured to detect temperature of liquid in the bottle body, and the display screen is configured to display the temperature of the liquid. The spray assembly is connected with a nozzle and a straw, where the straw extends into the bottle body.

Furthermore, the spray assembly further includes a casing and a guide tube. The casing is sealingly connected to the bottle body, and the guide tube is arranged in the casing. The temperature sensor and the display screen are respectively connected to the casing. A liquid injection port is defined on the casing, a first end of the guide pipe is communicated with the liquid injection port, and a second end of the guide pipe is communicated with the bottle body. The spray assembly further includes a first spray mechanism, where the first spray mechanism is located in the casing, and the nozzle and the straw are respectively communicated with the first spray mechanism.

Furthermore, the first spray mechanism includes a driving motor and a first pump body, the driving motor is connected with the first pump body through a transmission assembly, a first one-way valve is arranged at an inlet of the first pump body, and a second one-way valve is arranged at an outlet of the first pump body; the nozzle is connected to the outlet of the first pump body through the second one-way valve so that the liquid enters the first pump body from the inlet of the first pump body and flows out from the outlet of the first pump body to the nozzle.

Furthermore, the straw is a hose, a spring is sleeved on an outer ring of the hose, and the hose is connected to the inlet of the first pump body; a sealing ring is arranged on an outer ring of the casing, the casing is slidably connected with the bottle body through the sealing ring.

Furthermore, the spray assembly further includes a first cover and a second cover, and the first cover is buckled with the second cover. The driving motor, the transmission assembly, and the first pump body are located between the first cover and the second cover.

Furthermore, the transmission assembly includes a transmission gear, a connection gear, a connection rod, and a first piston. The transmission gear is connected to an output end of the driving motor, and the connection gear is rotatably connected with the first cover or the second cover. The transmission gear is engaged with the connection gear, an eccentric wheel is arranged on the connection gear, the connection rod is respectively connected to the eccentric wheel and the first piston for driving the first piston to slide back and forth with respect to an inner cavity of the first pump body.

Furthermore, a connection sleeve is arranged on the connection rod. A guide groove is defined on the connection sleeve, and the guide groove extends along a length direction of the connection rod. A positioning shaft is arranged on the eccentric wheel, the positioning shaft is inserted in the guide groove, and the positioning shaft is located at a rotation center of the connection gear.

Furthermore, the first one-way valve includes a first valve body, the first valve body is connected to the inlet of the first pump body, and a first baffle is arranged between the first valve body and the inlet of the first pump body so that the liquid flows into the inlet of the first pump body in a single direction. The first baffle includes a first mounting portion and a first stopper portion, the first stopper portion is connected to the first mounting portion, the first mounting portion is clamped between the first valve body and the first pump body, and the first stopper portion abuts against an opening of a channel of the first valve body.

Furthermore, the second one-way valve includes a second valve body, the second valve body is connected to the outlet of the first pump body, and a second baffle is arranged between the second valve body and the outlet of the first pump body so that the liquid flows out from the outlet of the first pump body in the single direction. The second baffle includes a second mounting portion and a second stopper portion, the second stopper portion is connected to the second mounting portion, the second mounting portion is clamped between the second valve body and the first pump body, and the second stopper portion abuts against the outlet of the first pump body, an elastic member is further arranged between the second stopper portion and the second valve body.

Furthermore, a battery is further arranged in the casing. A charging portion, a first switch, and a second switch are is arranged on the casing, and the charging portion, the first switch, and the second switch are respectively and electrically connected with the battery. The battery is located between the spray assembly and the guide tube, the first switch is configured to control the spray assembly, and the second switch is configured to control the display screen.

Furthermore, the spray assembly and the temperature display assembly are respectively located at opposite two ends of the bottle body. The temperature display assembly includes a base, the base is connected to the bottle body, the temperature sensor and the display screen are respectively connected to the base. The spray assembly further includes a connection member and a second spray mechanism, the connection member is connected to the bottle body, and the second spray mechanism is connected to the bottle body through the connection member, the nozzle and the straw are respectively communicated with the second spray mechanism.

Furthermore, the base includes a cavity and a cover body, and the cavity is connected to the cover body. The connection groove is defined on the cover body, and the temperature sensor is located at a bottom of the connection groove. A first connection port is defined at a first end of the bottle body. The bottle body and the cover body are connected to the connection groove through the first connection port.

Furthermore, an accommodating space is formed between the cavity and the cover body, and a battery is arranged in the accommodating space, a charging port and a power switch are respectively arranged on the cavity, where the charging port and the power switch are electrically connected with the battery.

Furthermore, the second spray mechanism includes a second pump body, a third one-way valve, and a fourth one-way valve, where the third one-way valve and the fourth one-way valve are respectively arranged at both ends of the second pump body, and a piston mechanism is further arranged on the second pump body. The third one-way valve is arranged at an inlet of the second pump body, and the fourth one-way valve is arranged at an outlet of the second pump body so that the liquid enters the second pump body through the inlet of the second pump body and flows out from the outlet of the second pump body.

Furthermore, the third one-way valve includes a third valve body, a first blocking head, and a conduit. The third valve body is arranged with respect to the inlet of the second bump body, the first blocking bead is matched with the third valve body, and the conduit is communicated with the second pump body, a first elastic member is arranged in the conduit and elastically abuts against the first blocking bead for driving the first blocking bead to abut against a channel of the third valve body.

Furthermore, the fourth one-way valve includes a fourth valve body, a valve core, and a second blocking bead, the fourth valve body is arranged with respect to the outlet of the second pump body, the valve core is arranged in the fourth valve body, and the second blocking bead is arranged with respect to the outlet of the second pump body. A second elastic member is arranged in the valve core and elastically abuts against the second blocking bead, a protrusion is arranged on an outer side of the valve core and is capable of abutting against the fourth valve body for driving the second blocking bead to abut against the outlet of the second pump body.

Furthermore, the piston mechanism includes a piston cylinder and a second piston. The piston cylinder is communicated with the second pump body, and the second piston is slidably connected with the piston cylinder. The second pump body is further rotatably connected with a handle, the handle is connected to the second piston through a piston rod, a third elastic member is further arranged in the piston cylinder, two ends of the third elastic member respectively abut against an end of the second piston and the piston cylinder.

Furthermore, the spray assembly further includes two protective covers, where the two protective covers are buckled with each other, and the second spray mechanism is wrapped in the two protective covers.

Furthermore, a second connection port is defined at a second end of the bottle body, the second connection port is connected to the connection member and abuts against the third valve body.

Furthermore, the bottle body is made of transparent material.

Compared with the prior art, beneficial effects of the embodiments of the present disclosure are as following.

The present disclosure provides the ear cleaning device, where the ear cleaning device provides the spray assembly and the temperature display assembly on the bottle body, which facilitates better cooperation between the temperature display assembly and the spray assembly. When in use, the temperature displayed on the display screen can be intuitively seen, so as to avoid the temperature being too high or too low when the spray assembly is operated. By arranging the temperature sensor in the bottle body, it can be ensured that the current temperature of the liquid in the bottle body can be displayed on the display screen in real time, which facilitates the control of the liquid temperature during the ear cleaning process.

### BRIEF DESCRIPTION OF THE DRAWINGS

In order to clearly explain technical solutions of embodiments of the present disclosure or in the related art, drawings used in the description of the embodiments or the related art are briefly described below. Obviously, the drawings as described below are merely some embodiments of the present disclosure. Based on these drawings, other drawings can be obtained by those skilled in the art without paying creative efforts.

FIG. 1 is a structural schematic diagram of a first embodiment of an ear cleaning device according to one embodiment of the present disclosure.

FIG. 2 is a structural schematic diagram of a first embodiment of a spray assembly according to one embodiment of the present disclosure.

FIG. 3 is another structural schematic diagram of the first embodiment of the spray assembly according to one embodiment of the present disclosure.

FIG. 4 is a structural schematic diagram of a spray mechanism according to one embodiment of the present disclosure.

FIG. 5 is another structural schematic diagram of the spray mechanism according to one embodiment of the present disclosure.

FIG. 6 is a structural schematic diagram of a first one-way valve and a second one-way valve according to one embodiment of the present disclosure.

FIG. 7 is a structural schematic diagram of a second embodiment of the ear cleaning device according to one embodiment of the present disclosure.

FIG. 8 is a structural schematic diagram of a temperature display assembly according to one embodiment of the present disclosure.

FIG. 9 is a structural schematic diagram of a bottle body according to one embodiment of the present disclosure.

FIG. 10 is a structural schematic diagram of a second embodiment of the spray assembly according to one embodiment of the present disclosure.

FIG. 11 is a partial enlarged diagram of portion A shown in FIG. 10.

Reference number: 100—ear cleaning device; 110—bottle body; 112—first connection port; 114—second connection port; 120—spray assembly; 121—casing; 1212—charging port; 1214—first switch; 1216—second switch; 1218—sealing ring; 122—battery; 123—guide tube; 124—temperature sensor; 125—liquid injection port; 126—display screen; 127—straw; 1272—spring; 128—protective cover; 129—connection member; 130—first spray mechanism; 131—driving motor; 132—first pump body; 133—transmission assembly; 1332—transmission gear; 1334—connection gear; 1334a—eccentric wheel; 1334b—positioning shaft; 1336—connection rod; 1336a—connection sleeve; 1336b—guide groove; 1338—first piston; 134—first one-way valve; 1344—first valve body; 1346—first baffle; 1346a—first mounting portion; 1346b—first stopper portion; 135—second one-way valve; 1354—second valve body; 1356—second baffle; 1356a—second mounting portion; 1356b—second stopper portion; 1358—elastic member; 136—first cover; 138—second cover; 142—base; 1422—cavity body; 1424—cover body; 1424a—connection groove; 146—power switch; 150—second spray mechanism; 152—second pump body; 154—third one-way valve; 1542—third valve body; 1544—first blocking bead; 1546—conduit; 1548—first elastic member; 156—fourth one-way valve; 1562—fourth valve body; 1564—valve core; 1564a—protrusion; 1566—second blocking bead; 1568—second elastic member; 158—piston mechanism; 1582—piston cylinder; 1584—second piston; 1586—handle; 1588—piston rod; 1589—third elastic member; 160—nozzle; 162—protective shield; 140—temperature display assembly.

### DETAILED DESCRIPTION

In order to make objectives, technical solutions, and advantages of embodiments of the present disclosure clearer, technical solutions in the embodiments of the present disclosure are clearly and completely described below with reference to the accompanying drawings in the embodiments of the present disclosure. Obviously, the described embodiments are some embodiments of the present disclosure, rather than all embodiments. Assemblies of the embodiments of the present disclosure, which are generally described and illustrated herein, may be arranged and designed in a variety of different configurations.

Therefore, following detailed description of the embodiments of the present disclosure provided in the accompanying drawings is not intended to limit the scope of the present disclosure, but merely represents a selected embodiment of the present disclosure. All other embodiments obtained by a person of ordinary skill in the art based on the embodiments of the present disclosure without creative efforts shall fall within the protection scope of the present disclosure.

It should be noted that similar reference numerals and letters represent similar items in following drawings, and therefore, once a certain item is defined in one drawing, it is not necessary to further define and interpret it in the subsequent drawings.

In the description of the present disclosure, it should be noted that the orientation or positional relationship indicated by the terms “inner”, “outside”, etc. is based on the orientation or positional relationship shown in the drawings, or the orientation or positional relationship of customary placement when products related to the present disclosure is used, and is merely intended to facilitate the description of the present disclosure and the simplified description, rather than indicating or implying that the referred device or element must have a specific orientation, so as to be constructed and operated in a specific orientation, and therefore cannot be understood as a limitation to the present disclosure. In addition, terms such as “first” and “second” are merely used for distinguishing descriptions, and cannot be understood as indicating or implying relative importance.

In the description of the present disclosure, it should also be noted that the terms “arrange” and “connect” should be construed broadly unless expressly specified and defined otherwise, for example, may be fixedly connecting, detachably connecting, or integrally connecting; may be mechanically connecting or electrically connecting; may be directly connecting or indirectly connecting by means of an intermediate medium, and may be internally communicating between two elements. For a person of ordinary skill in the art, the specific meaning of the above terms in the present disclosure may be specifically understood.

Based on a problem that patients may easily feel discomfort during auditory canal treatment or cleaning when temperature of liquid is too high or too low, the embodiments of the present disclosure provide following technical solutions to solve the above problem.

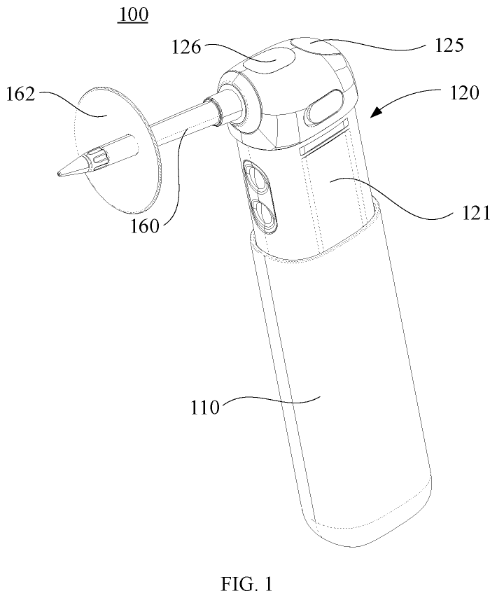

Referring to FIGS. 1, 2, 7, and 8, an ear cleaning device 100 is illustrated, the ear cleaning device 100 includes a bottle body 110, a spray assembly 120, and a temperature display assembly 140. The spray assembly 120 and the temperature display assembly 140 are arranged on the bottle body 110. The temperature display assembly 140 includes a temperature sensor 124 and a display screen 126, and the display screen 126 is electrically connected with the temperature sensor 124. The temperature sensor 124 is arranged in the bottle body 110 and is configured to detect the temperature of the liquid in the bottle body 110, and the display screen 126 is configured to display the temperature of the liquid. The spray assembly 120 is connected to a nozzle 160 and a straw 127, and the straw 127 extends into the bottle body 110.

Specifically, the bottle body 110 is configured to contain the liquid for cleaning the ear, when in use, the nozzle 160 is connected to the spray assembly 120 and the straw 127 extends into the bottle body 110 for facilitating required liquid spray operation. During the required liquid spray operation, the temperature of the liquid may be detected by the temperature sensor 124 arranged in the bottle body 110 and displayed on the display screen 126. The temperature sensor 124 may be in a form of a temperature probe needle or a temperature probe head, which is not specifically limited herein.

The ear cleaning device 100 provided by the embodiment provides the spray assembly 120 and the temperature display assembly 140 on the bottle body 110, which facilitates better cooperation between the temperature display assembly 140 and the spray assembly 120. When in use, the temperature displayed on the display screen 126 can be intuitively seen, so as to avoid the temperature being too high or too low when the spray assembly 120 is operated. By arranging the temperature sensor 124 in the bottle body 110, it can be ensured that the current temperature of the liquid in the bottle body 110 can be displayed on the display screen 126 in real time, which facilitates the control of the liquid temperature during the ear cleaning process.

Referring to FIGS. 1, 2, and 3, the spray assembly 120 further includes a casing 121 sealed with the bottle body 110, a guide tube 123 arranged in the casing 121, the temperature sensor 124 and the display screen 126 are respectively connected to the casing 121. A liquid injection port is further defined in the casing 121, a first end of the guide pipe 123 is connected to the liquid injection port 125, and a second end of the guide pipe 123 is connected to the bottle body 110. The spray assembly 120 comprises a first spray mechanism 130 located in the casing 121, and the nozzle 160 and the straw 127 are respectively communicated with the first spray mechanism 130.

Specifically, by arranging the guide tube 123 communicated with the bottle body 110 in the casing 121, and arranging the liquid injection port 125 communicated with the guide tube 123 on the casing 121, when the ear cleaning device 100 needs to be in use, the liquid is injected from the liquid injection port 125 and flows into the bottle body 110 along the guide tube 123. It can be understood that, in order to prevent the liquid from flowing out of the liquid injection port 125, a sealing cover may be arranged on the liquid injection port 125. In addition, by arranging the first spray mechanism 130 in the casing 121, the liquid spray operation is realized through the first spray mechanism 130.

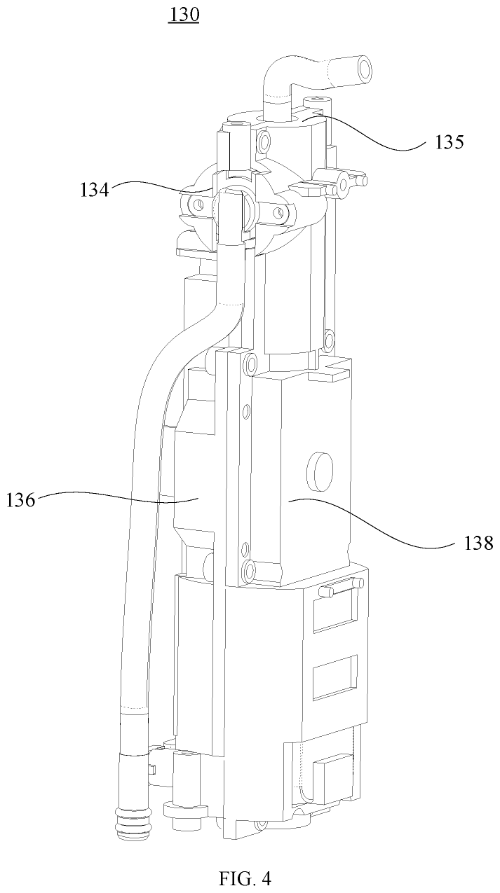

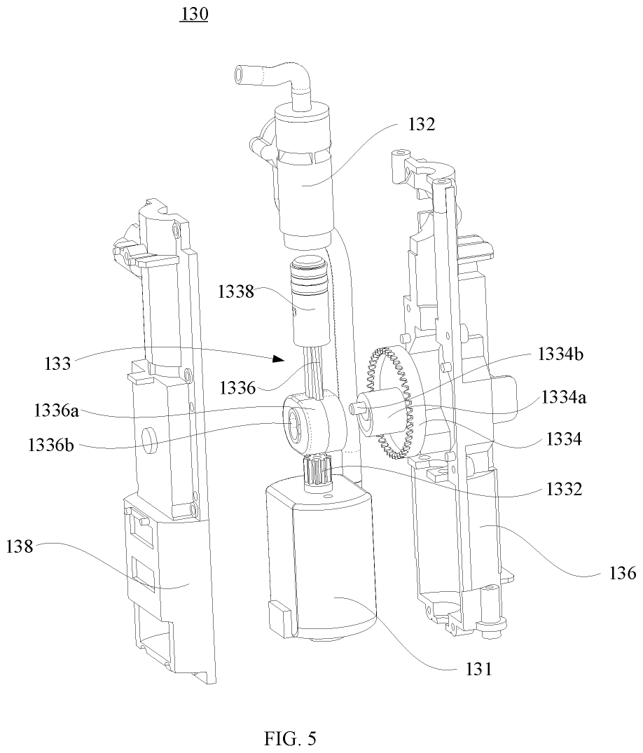

When the ear cleaning device 100 is electric, referring to FIGS. 4 and 5, the first spray mechanism 130 includes a driving motor 131 and a first pump body 132, the driving motor 131 is connected to the first pump body 132 through a transmission assembly 133. A first one-way valve 134 is arranged at an inlet of the first pump body 132, and a second one-way valve 135 is arranged at an outlet of the first pump body 132. The nozzle 160 is communicated with the outlet of the first pump body 132 through the second one-way valve 135 so that the liquid enters the first pump body 132 from the inlet of the first pump body 132 and flows out from the outlet of the first pump body 132 to the nozzle 160.

Specifically, during the liquid spraying operation, the driving motor 131 drives the transmission assembly 133 to move, and then the transmission assembly 133 is cooperated with the first pump body 132 for sucking and spraying the liquid. During the liquid sucking action, the second one-way valve 135 is closed, and the first one-way valve 134 is opened, therefore, the liquid flows from the first one-way valve 134 into the first pump body 132. During the liquid spraying action, the second one-way valve 135 is opened, and the first one-way valve 134 is closed, therefore, the liquid is sprayed from the second one-way valve 135. At this time, the sprayed liquid flows along the nozzle 160, and finally sprays towards an area to be cleaned.

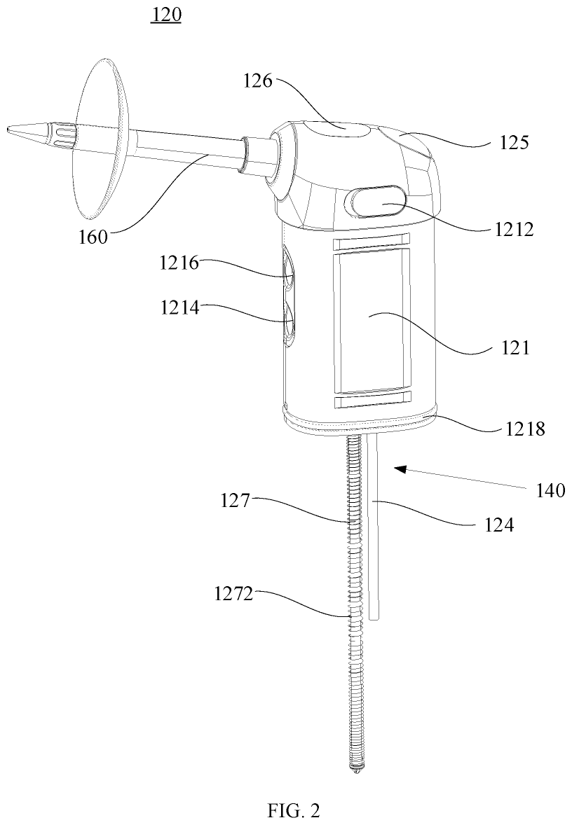

In one embodiment, as illustrated in FIG. 2, the straw 127 may be a hose, a spring 1272 is sleeved on an outer ring of the hose, and the hose is connected to the inlet of the first pump body 132. A sealing ring 1218 is arranged on an outer ring of the casing 121, the casing 121 is slidably connected with the bottle body 110 through the sealing ring 1218.

Specifically, by arranging the hose extending into the bottle body 110 on the casing 121 and the spring 1272 sleeved on the outer ring of the hose, when the casing 121 and the bottle body 110 slide through the sealing ring 1218, if the space in the bottle body 110 becomes smaller, the spring 1272 may be compressed to prevent the sliding of the bottle body 110 from being hindered, at the same time, the stability of the position of the hose can be ensured. Similarly, if the space in the bottle body 110 becomes larger, the spring 1272 may support the hose to a certain extent, so as to ensure that the end of the hose is at the bottom of the bottle body 110, thereby ensuring smoothly liquid sucking.

As illustrated in FIGS. 1 and 5, the spray assembly 120 further includes a first cover 136 and a second cover 138 that are buckled with each other, the driving motor 131, the transmission assembly 133, and the first pump body 132 are located between the first cover 136 and the second cover 138.

Specifically, the first cover 136 and the second cover 138 that are buckled with each other may support and protect the driving motor 131, the transmission assembly 133, and the pump body. When arranging the spray assembly 120 in the casing 121, the spray assembly 120 may be directly arranged as a sub-module that is assembled with the casing 121, there is no need to use the casing 121 for positioning and installation, which is beneficial to improve the assembly efficiency, when the spray assembly 120 is cooperatively connected with the casing 121, the first cover 136 and/or the second cover 138 is required to be connected with the casing 121.

As illustrated in FIG. 5, the transmission assembly 133 includes a transmission gear 1332 connected to an output end of the driving motor 131, a connection gear 1334 rotatably connected with the first cover 136 or the second cover 138, a connection rod 1336, and a first piston 1338. The transmission gear 1332 is engaged with the connection gear 1334, an eccentric wheel 1334a is arranged on the connection gear 1334, the connection rod 1336 is respectively connected to the eccentric wheel 1334a and the first piston 1338 for driving the first piston 1338 to slide back and forth with respect to an inner cavity of the first pump body 132.

According to the above embodiment, when the driving motor 131 rotates, the driving motor 131 drives the transmission gear 1332 to rotate, and the transmission gear 1332 drives the connection gear 1334 to rotate with respect to the first cover 136 or the second cover 138. At this time, the eccentric wheel 1334a arranged on the connection gear 1334 eccentrically rotates, the connection rod 1336 is respectively connected with the eccentric wheel 1334a and the first piston 1338, then the first piston 1338 moves back and forth with respect to the inner cavity of the first pump body 132 through driving the connection rod 1336 by the eccentric wheel 1334a, and then through the cooperation of the first one-way valve 134 and the second one-way valve 135, the required liquid sucking and spraying actions are realized.

As illustrated in FIG. 5, a connection sleeve 1336a is sleeved on the connection rod 1336, a guide groove 1336b is defined on the connection sleeve 1336a, where the guide groove 1336b extends along a length direction of the connection rod 1336, a positioning shaft 1334b is arranged on the eccentric wheel 1334a and is further inserted in the guide groove 1336b, and the positioning shaft 1334b is located at a rotation center of the connection gear 1334.

Specifically, by arranging the connection sleeve 1336a on the connection rod 1336, the connection sleeve 1336a is arranged on the outer ring of the eccentric wheel 1334a, so as to move toward or away from the pump body by following the eccentric wheel 1334a. At the same time, through a joint limiting action of the positioning shaft 1334b and the first pump body 132, the connection rod 1336 straightly moves back and forth between the first pump body 132 and the positioning shaft 1334b, which is beneficial to ensure the stability during transmission.

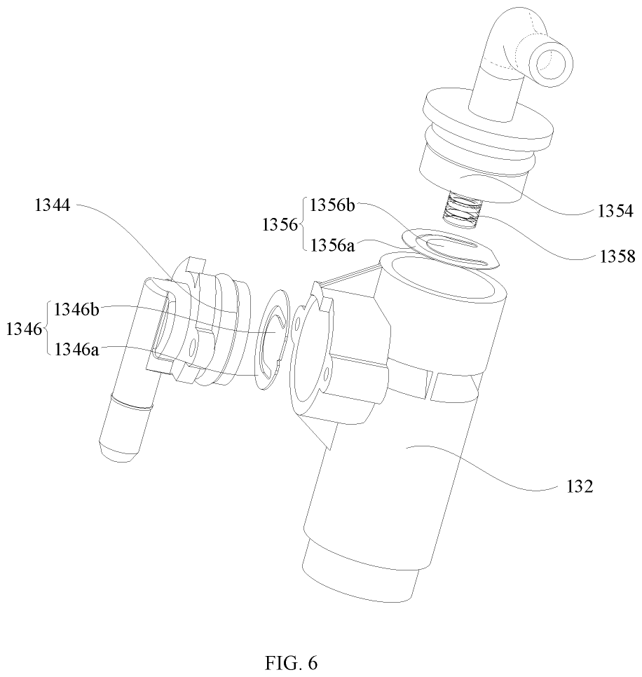

Referring to FIGS. 4 and 6, the first one-way valve 134 includes a first valve body 1344, the first valve body 1344 is connected to the inlet of the first pump body 132, and a first baffle 1346 is arranged between the first valve body 1344 and the inlet of the first pump body 132 so that the liquid only flows into the inlet of the first pump body 132 in a single direction from. The first baffle 1346 includes a first mounting portion 1346a and a first stopper portion 1346b connected to the first mounting portion 1346b, the first mounting portion 1346a is clamped between the first valve body 1344 and the first pump body 132, and the first stopper portion 1346b abuts against an opening of a channel of the first valve body 1344.

Specifically, in an initial state, the first stopper portion 1346b abuts against the channel of the first valve body 1344 for blocking the inlet of the first pump body 132. Under the action of the first piston 1338, a negative pressure is generated in the first pump body 132, the first stopper portion 1346b is deformed by force, thus, a gap is formed between the first stopper portion 1346b and the opening of the channel of the first valve body 1344, as a result, the first one-way valve 134 and the inlet of the first pump body 132 are communicated, so as to suck the liquid into the first pump body 132, which provides a basis for the liquid spraying action.

As illustrated in FIGS. 4 and 6, the second one-way valve 135 includes a second valve body 1354, the second valve body 1354 is connected to the outlet of the first pump body 132, and a second baffle 1356 is arranged between the second valve body 1354 and the outlet of the first pump body 132 so that the liquid may only flow out from the outlet of the first pump body 132 in the single direction. The second baffle includes a second mounting portion 1356a and a second stopper portion 1356b connected to the second mounting portion 1356a, the second mounting portion 1356a is clamped between the second valve body 1354 and the first pump body 132, and the second stopper portion 1356b abuts against the outlet of the first pump body 132, and an elastic member 1358 is further arranged between the second stopper portion 1356b and the second valve body 1354.

Specifically, when the first piston 1338 operates, a negative pressure is generated in the first pump body 132, the second stopper portion 1356b is under force, abuts against, and closely contacts the outlet of the first pump body 132, so as to ensure the sealing performance of the second stopper portion 1356b and the outlet of the first pump body 132, thereby avoiding sucking liquid due to air leakage. When the first piston 1338 operates, the pressure in the first pump body 132 increases, the first stopper portion 1346b is under force, abuts against, and closely contacts the opening of the channel of the first valve body 1344, so as to ensure the blocking capability of the first one-way valve 134. At this time, the second stopper portion 1356b is deformed by the reverse force, a gap is formed between the second stopper portion 1356b and the outlet of the first pump body 132, so that the second one-way valve 135 and the outlet of the first pump body 132 are connected, the liquid is thus sprayed. When the liquid spraying action is completed, the liquid sucking action is performed, the second stopper portion 1356b can be quickly reset by the force of the elastic member 1358, so as to ensure the effect of the liquid suction operation.

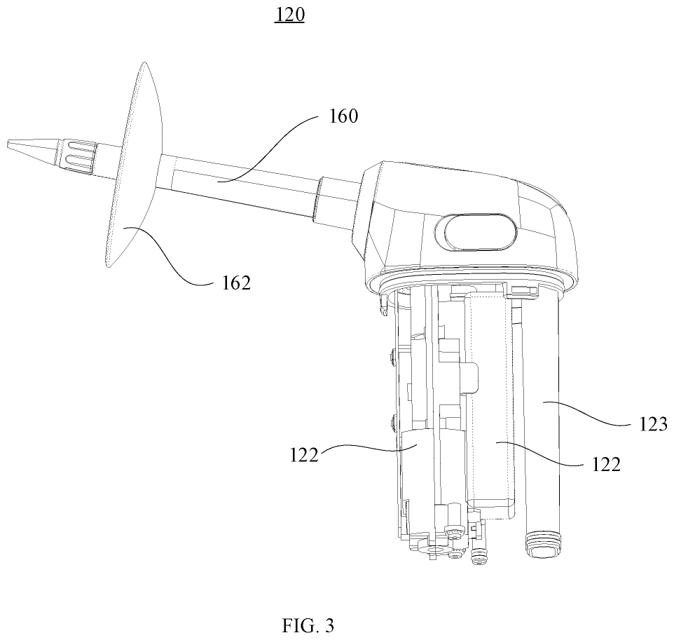

In one embodiment, as illustrated in FIGS. 2 and 3, a battery 122 is further arranged in the casing 121, and a charging port 1212 electrically connected with the battery 122, a first switch 1214, and a second switch 1216 are respectively arranged on the casing 121, the battery 122 is located between the spray assembly 120 and the guide tube 123, the first switch 1214 is configured to control the spray assembly 120, and the second switch 1216 is configured to control the display screen 126.

Specifically, by arranging the battery 122, the required electric power is provided for the operation of the ear cleaning device 100. In addition, by arranging the charging port 1212, it is beneficial to realize the supplement of electric energy. It can be understood that, in order to prevent the charging port 1212 from being affected by the outside world, protective covers 128 may be provided at the charging port 1212. At the same time, by arranging the first switch 1214 and the second switch 1216, the spray assembly 120 and the display screen 126 may be respectively controlled, which is beneficial to save electric energy and improve the diversification of the control.

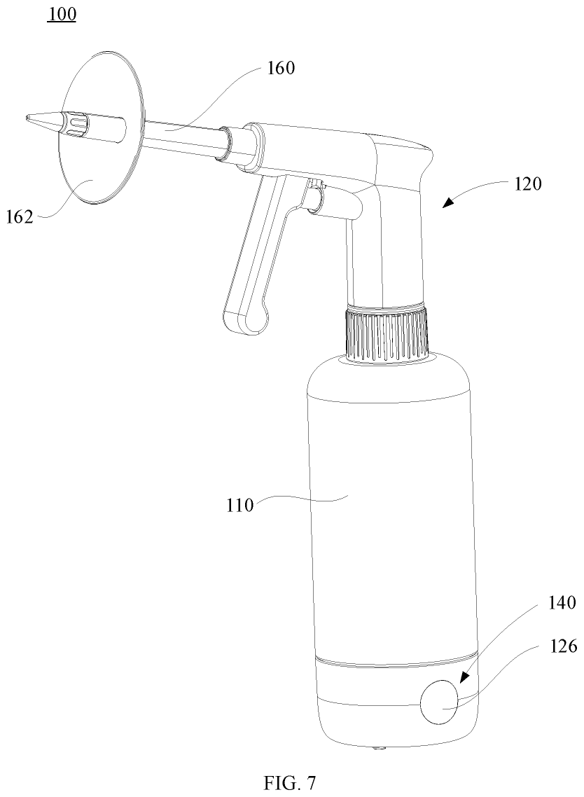

When the ear cleaning device 100 is in a manual form, that is, the ear cleaning device 100 is manually operated, as illustrated in FIGS. 7, 8, and 10, the spray assembly 120 and the temperature display assembly 140 are respectively located at opposite two ends of the bottle body 110. The temperature display assembly 140 includes a base 142 connected with the bottle body 110, the temperature sensor 124 and the display screen 126 are respectively connected to the base 142. the spray assembly 120 further includes a connection member 129 connected with the bottle body 110, and a second spray mechanism 150 connected with the bottle body 110 through the connection member 129, the nozzle 160 and the straw 127 are respectively connected with the second spray mechanism 150.

Specifically, when in use, the bottle body 110 is connected with the base 142, and the spray assembly 120 is removed from the bottle body 110, so as to fill the bottle body 110 with the liquid, when the liquid injection is completed, the spray assembly 120 can be reconnected with the bottle body 110. At this time, the required liquid spraying operation can be realized by the liquid spray mechanism 150.

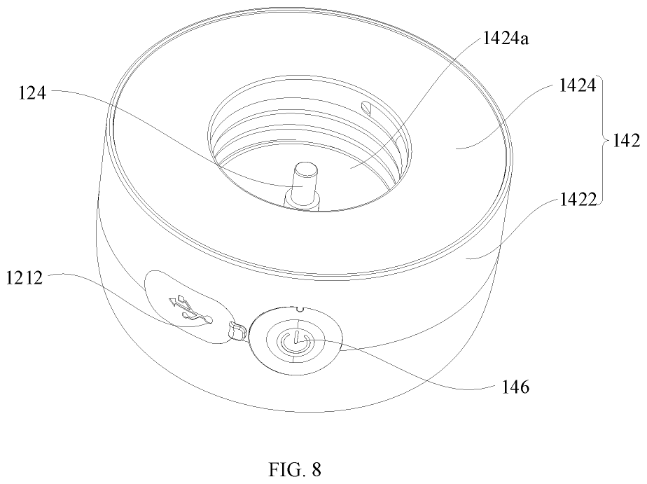

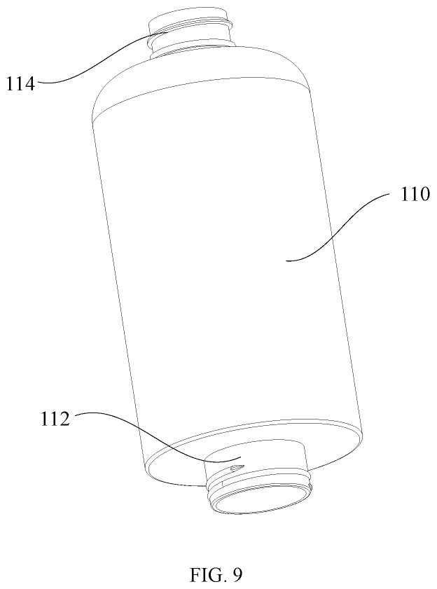

As illustrated in FIGS. 8 and 9, the base 142 includes a cavity body 1422 and a cover body 1424 that are connected with each other. The cover body 1424 is provided with a connection groove 1424a, and the temperature sensor 124 is located at the bottom of the connection groove 1424a; an end of the bottle body 110 is provided with a first connection port 112, so that the bottle body 110 and the cover body 1424 are connected with the connection groove 1424a through the first connection port 112.

Specifically, the first connection port 112 is provided with an external thread, and a side wall surface of the connection groove 1424a is provided with an internal thread correspondingly, so that the bottle body 110 and the cover body 1424 are connected by thread. It can be understood that the cross sections of the bottle body 110 and the base 142 are equal diameter circles, and an integral cylindrical structure is formed after the bottle body 110 and the base 142 are connected, which is beneficial to improve the reliability of the overall connection and ensures the smooth transition of the connection. In addition, by arranging the temperature sensor 124 at the bottom of the connection groove 1424a, it is beneficial to ensure the reliable setting of the temperature sensor 124, and to form the required connection relationship after the bottle body 110 and the cover body 1424 are connected.

In one embodiment, referring to FIG. 8, an accommodating space is formed between the cavity body 1422 and the cover body 1424, and a battery (not shown in FIG. 8) is arranged in the accommodating space, a charging port 1212 and a power switch 146 electrically connected with the battery are arranged on the cavity body 1422.

Specifically, by arranging the battery 122 in the accommodating space and arranging the charging port 1212 connected with the battery on the cavity body 1422, charging can be performed when the power is insufficient, which is beneficial to the utilization rate of energy. In addition, by arranging the power switch 146 electrically connected with the battery on the cavity body 1422, the power can be turned off when the ear cleaning device 100 is not in use and turned on when the ear cleaning device 100 is in use, which is beneficial to save energy, and prolong the service life of the battery and the temperature sensor 124.

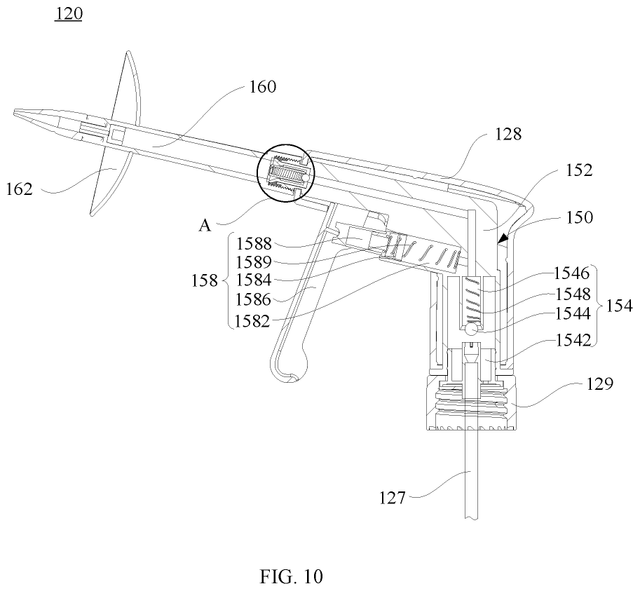

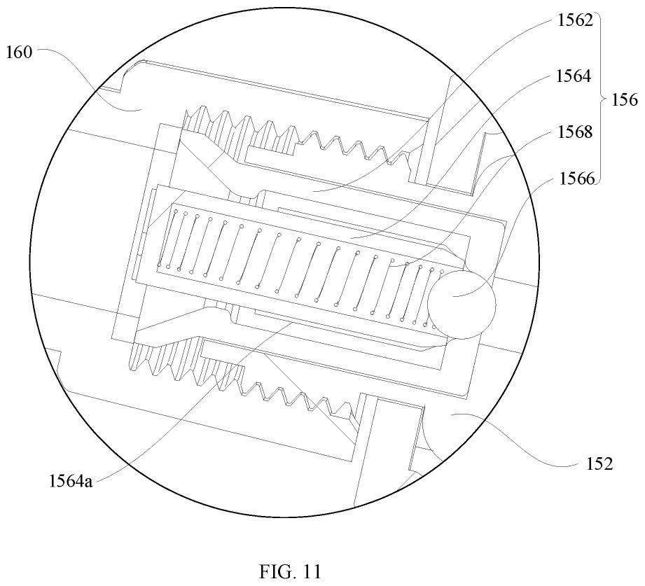

Referring to FIGS. 10 and 11, the second spray mechanism 150 includes a second pump body 152, a third one-way valve 154, and a fourth one-way valve 156 respectively arranged at both ends of the second pump body 152. The second pump body 152 is further provided with a piston mechanism 158, the third one-way valve 154 is arranged at an inlet of the second pump body 152, and the fourth one-way valve 156 is arranged at an outlet of the second pump body 152, so that the liquid flows from the inlet of the second pump body 152 and enters the second pump body 152, and flows out from the outlet of the second pump body 152.

Specifically, by arranging the third one-way valve 154 and the fourth one-way valve 156 at both ends of the second pump body 152, when the piston mechanism 158 performs the liquid suction action, a negative pressure is generated in the second pump body 152, so that the third one-way valve 154 is opened, the fourth one-way valve 156 is closed, and the liquid flows into the second pump body 152 through the third one-way valve 154. When the piston mechanism 158 performs the liquid spraying action, the pressure in the second pump body 152 increases, so that the third one-way valve 154 is closed, and the fourth one-way valve 156 is opened, the liquid inhaled into the second pump body 152 is sprayed out through the fourth one-way valve 156, thereby achieving the effect of washing.

As illustrated in FIGS. 9 and 10, the third one-way valve 154 includes a third valve body 1542 corresponding to the inlet of the second bump body 152, a first blocking bead 1544 cooperated with the third valve body 1542, and a conduit 1546 connected with the second pump body 152, a first elastic member 1548 is arranged in the conduit 1546, the first elastic member 1548 elastically abuts against the first blocking bead 1544, thus, the first blocking bead 1544 is driven to abut against the channel of the third valve body 1542.

Specifically, in the initial state, the first blocking bead 1544 abuts against the channel of the third valve body 1542 under the elastic resisting force of the first elastic member 1548, so that the third one-way valve 154 is in a closed state. The other end of the bottle body 110 is provided with a second connection port 114, the second connection port 114 is connected with the connection member 129 and abuts against the third valve body 1542, so that the third valve body 1542 is limited between the connection member 129 and the second connection port 114, and the connection between the second pump body 152 and the third valve body 1542 is fixed. The channel of the third valve body 1542 is provided with a bell mouth, the first blocking bead 1544 can be closely fitted with the channel of the third valve body 1542. When the negative pressure is generated in the second pump body 152, the second pump body 152 overcomes the elastic force of the first elastic member 1548 to do work, so that the first blocking bead 1544 is far away from the channel of the third valve body 1542, so as to ensure that the liquid flows into the second pump body 152 smoothly. When the suction action is completed, the first blocking bead 1544 abuts against the channel of the third valve body 1542 again by the force of the first elastic member 1548.

As illustrated in FIG. 11, the fourth one-way valve 156 includes a fourth valve body 1562 corresponding to the outlet of the second pump body 152, a valve core 1564 arranged in the fourth valve body 1562, and a second blocking bead 1566 corresponding to the outlet of the second pump body 152, the valve core 1564 is provided with a second elastic member 1568, the second elastic member 1568 elastically abuts against the second blocking bead 1566, and the outer side of the valve core 1564 is provided with a protrusion 1564a, the protrusion 1564a is capable of abutting against the fourth valve body 1562, so that the second blocking bead 1566 is driven to abut against the outlet of the second pump body 152.

Specifically, in the initial state, the second blocking bead 1566 abuts against the outlet of the second pump body 152 by the elastic resisting force of the second elastic member 1568, so that the third one-way valve 154 is in a closed state. When the negative pressure is generated in the second pump body 152, the second blocking bead 1566 contacts the outlet of the second pump body 152 more closely, thereby ensuring the negative pressure strength in the second pump body 152. During the liquid spraying operation, the pressure in the second pump body 152 increases, and overcomes the elastic force of the second elastic member 1568 to do work, so that the second blocking bead 1566 is far away from the outlet of the second pump body 152, and the liquid in the second pump body 152 flows out along the gap between the valve core 1564 and the second valve body 1354. In addition, by arranging the protrusion 1564a that can abut against the second valve body 1354 on the outside of the valve core 1564, the position of the valve core 1564 can be limited, thereby ensuring the stability of the third one-way valve 154 when in use. When the liquid spraying action is completed, the second blocking bead 1566 abuts against the outlet of the second pump body 152 again by the force of the second elastic member 1568. The nozzle 160 is connected with the outlet of the second pump body 152 by thread, when the nozzle 160 is connected with the second pump body 152, a connection position between the nozzle 160 and the second pump body 152 abuts against the fourth valve body 1562, so that the fourth valve body 1562 is limited between the second pump body 152 and the nozzle 160, thereby ensuring the stability of the connection.

As shown in FIG. 10, the piston mechanism 158 includes a piston cylinder 1582 that is connected with the second pump body 152 and a second piston 1584 that is slidably connected with the piston cylinder 1582. The second pump body 152 is further rotatably connected with a handle 1586, the handle 1586 is connected with the second piston 1584 through the piston rod 1588, a third elastic member 1589 is further arranged in the piston cylinder 1582, two ends of the third elastic member 1589 abut against an end of the second piston 1584 and the piston cylinder 1582 respectively.

Specifically, through the piston cylinder 1582 connected with the second pump body 152 and the second piston 1584 slidably connected with the piston cylinder 1582, when the second piston 1584 slides away from the second pump body 152, the negative pressure is generated in the second pump body 152 for liquid suction. When the second piston 1584 slides in a direction close to the second pump body 152, the pressure in the second pump body 152 increases for liquid spraying. In addition, the two ends of the third elastic member 1589 abut against one end of the second piston 1584 and the piston cylinder 1582 respectively, after the handle 1586 drives the second piston 1584 to slide in the direction close to the second pump body 152, the handle 1586 is restored to the initial state by the elastic force of the third elastic member 1589, so that the handle 1586 can apply the force on the second piston 1584 and drive the second piston 1584 to slide next time.

As illustrated in FIG. 10, the spray assembly 120 further includes two protective covers 128 that are buckled with each other, and the second spray mechanism 150 is wrapped in the protective covers 128. By adopting the above form, the outer surface of the spray assembly 120 is smoother, and the stability when in use can be ensured.

As illustrated in FIGS. 1 and 7, the nozzle 160 is further provided with a protective shield 162, the protective shield 162 is a curved surface structure, and a concave surface is directed to the nozzle, which can play a certain protective role when spraying liquid, and prevent the liquid from splashing everywhere. It can be understood that the protective shield 162 can be made of transparent plastic, thereby avoiding blocking the line of sight during use.

In one optional embodiment, the bottle body 110 is made of transparent material. For example, the bottle body 110 can be made of transparent plastic or transparent glass. Thus, it is convenient to observe the amount of the solution in the bottle body 110, so as to improve the operability during use, and the solution can be replenished in time as required.

### INDUSTRIAL APPLICABILITY

The ear cleaning device 100 provided in the present disclosure may display the temperature of the current liquid in real time, which facilitates the control of the temperature of the liquid during the washing process.

Technical features in the above embodiments can be combined arbitrarily. For concise description, not all possible combinations of the technical features in the above embodiments are described. However, any combination of the technical features described in the above embodiments should fall within the scope of the present disclosure, as long as no conflict occurs between the combined technical features.

What is claimed is:

1. An ear cleaning device, comprising:
a bottle body;
a spray assembly; and
a temperature display assembly;
wherein the spray assembly and the temperature display assembly are arranged on the bottle body; the temperature display assembly comprises a temperature sensor and a display screen, the display screen is electrically connected with the temperature sensor; the temperature sensor is arranged in the bottle body and is configured to detect temperature of liquid in the bottle body, and the display screen is configured to display the temperature of the liquid; the spray assembly is connected with a nozzle and a straw, where the straw extends into the bottle body.

2. The ear cleaning device according to claim 1, wherein the spray assembly further comprises a casing and a guide tube; the casing is sealingly connected to the bottle body, and the guide tube is arranged in the casing; the temperature sensor and the display screen are respectively connected to the casing; a liquid injection port is defined on the casing, a first end of the guide pipe is communicated with the liquid injection port, and a second end of the guide pipe is communicated with the bottle body; the spray assembly further comprises a first spray mechanism, where the first spray mechanism is located in the casing, and the nozzle and the straw are respectively communicated with the first spray mechanism.

3. The ear cleaning device according to claim 2, wherein the first spray mechanism comprises a driving motor and a first pump body, the driving motor is connected with the first pump body through a transmission assembly, a first one-way valve is arranged at an inlet of the first pump body, and a second one-way valve is arranged at an outlet of the first pump body; the nozzle is connected to the outlet of the first pump body through the second one-way valve so that the liquid enters the first pump body from the inlet of the first pump body and flows out from the outlet of the first pump body to the nozzle.

4. The ear cleaning device according to claim 3, wherein the straw is a hose, a spring is sleeved on an outer ring of the hose, and the hose is connected to the inlet of the first pump body; a sealing ring is arranged on an outer ring of the casing, the casing is slidably connected with the bottle body through the sealing ring.

5. The ear cleaning device according to claim 3, wherein the spray assembly further comprises a first cover and a second cover, and the first cover is buckled with the second cover; the driving motor, the transmission assembly, and the first pump body are located between the first cover and the second cover.

6. The ear cleaning device according to claim 5, wherein the transmission assembly comprises a transmission gear, a connection gear, a connection rod, and a first piston; the transmission gear is connected to an output end of the driving motor, and the connection gear is rotatably connected with the first cover or the second cover; the transmission gear is engaged with the connection gear, an eccentric wheel is arranged on the connection gear, the connection rod is respectively connected to the eccentric wheel and the first piston for driving the first piston to slide back and forth with respect to an inner cavity of the first pump body.

7. The ear cleaning device according to claim 6, wherein a connection sleeve is arranged on the connection rod; a guide groove is defined on the connection sleeve, and the guide groove extends along a length direction of the connection rod; a positioning shaft is arranged on the eccentric wheel, the positioning shaft is inserted in the guide groove, and the positioning shaft is located at a rotation center of the connection gear.

8. The ear cleaning device according to claim 3, wherein the first one-way valve comprises a first valve body, the first valve body is connected to the inlet of the first pump body, and a first baffle is arranged between the first valve body and the inlet of the first pump body so that the liquid flows into the inlet of the first pump body in a single direction; the first baffle comprises a first mounting portion and a first stopper portion, the first stopper portion is connected to the first mounting portion, the first mounting portion is clamped between the first valve body and the first pump body, and the first stopper portion abuts against an opening of a channel of the first valve body.

9. The ear cleaning device according to claim 8, wherein the second one-way valve comprises a second valve body, the second valve body is connected to the outlet of the first pump body, and a second baffle is arranged between the second valve body and the outlet of the first pump body so that the liquid flows out from the outlet of the first pump body in the single direction; the second baffle comprises a second mounting portion and a second stopper portion, the second stopper portion is connected to the second mounting portion, the second mounting portion is clamped between the second valve body and the first pump body, and the second stopper portion abuts against the outlet of the first pump body, an elastic member is further arranged between the second stopper portion and the second valve body.

10. The ear cleaning device according to claim 2, wherein a battery is further arranged in the casing; a charging portion, a first switch, and a second switch are is arranged on the casing, and the charging portion, the first switch, and the second switch are respectively and electrically connected with the battery; the battery is located between the spray assembly and the guide tube, the first switch is configured to control the spray assembly, and the second switch is configured to control the display screen.

11. The ear cleaning device according to claim 1, wherein the spray assembly and the temperature display assembly are respectively located at opposite two ends of the bottle body; the temperature display assembly comprises a base, the base is connected to the bottle body, the temperature sensor and the display screen are respectively connected to the base; the spray assembly further comprises a connection member and a second spray mechanism, the connection member is connected to the bottle body, and the second spray mechanism is connected to the bottle body through the connection member, the nozzle and the straw are respectively communicated with the second spray mechanism.

12. The ear cleaning device according to claim 11, wherein the base comprises a cavity and a cover body, and the cavity is connected to the cover body; the connection groove is defined on the cover body, and the temperature sensor is located at a bottom of the connection groove; a first connection port is defined at a first end of the bottle body; the bottle body and the cover body are connected to the connection groove through the first connection port.

13. The ear cleaning device according to claim 12, wherein an accommodating space is formed between the cavity and the cover body, and a battery is arranged in the accommodating space, a charging port and a power switch are respectively arranged on the cavity, where the charging port and the power switch are electrically connected with the battery.

14. The ear cleaning device according to claim 12, wherein the second spray mechanism comprises a second pump body, a third one-way valve, and a fourth one-way valve, where the third one-way valve and the fourth one-way valve are respectively arranged at both ends of the second pump body, and a piston mechanism is further arranged on the second pump body; the third one-way valve is arranged at an inlet of the second pump body, and the fourth one-way valve is arranged at an outlet of the second pump body so that the liquid enters the second pump body through the inlet of the second pump body and flows out from the outlet of the second pump body.

15. The ear cleaning device according to claim 14, wherein the third one-way valve comprises a third valve body, a first blocking head, and a conduit; the third valve body is arranged with respect to the inlet of the second bump body, the first blocking bead is matched with the third valve body, and the conduit is communicated with the second pump body, a first elastic member is arranged in the conduit and elastically abuts against the first blocking bead for driving the first blocking bead to abut against a channel of the third valve body.

16. The ear cleaning device according to claim 15, wherein the fourth one-way valve comprises a fourth valve body, a valve core, and a second blocking bead, the fourth valve body is arranged with respect to the outlet of the second pump body, the valve core is arranged in the fourth valve body, and the second blocking bead is arranged with respect to the outlet of the second pump body; a second elastic member is arranged in the valve core and elastically abuts against the second blocking bead, a protrusion is arranged on an outer side of the valve core and is capable of abutting against the fourth valve body for driving the second blocking bead to abut against the outlet of the second pump body.

17. The ear cleaning device according to claim 16, wherein the piston mechanism comprises a piston cylinder and a second piston; the piston cylinder is communicated with the second pump body, and the second piston is slidably connected with the piston cylinder; the second pump body is further rotatably connected with a handle, the handle is connected to the second piston through a piston rod, a third elastic member is further arranged in the piston cylinder, two ends of the third elastic member respectively abut against an end of the second piston and the piston cylinder.

18. The ear cleaning device according to claim 11, wherein the spray assembly further comprises two protective covers, where the two protective covers are buckled with each other, and the second spray mechanism is wrapped in the two protective covers.

19. The ear cleaning device according to claim 15, wherein a second connection port is defined at a second end of the bottle body, the second connection port is connected to the connection member and abuts against the third valve body.

20. The ear cleaning device according to claim 1, wherein the bottle body is made of transparent material.
